(19) United States
(12) Patent Application Publication
(10) Pub. No.: US 2016/0289141 A1
(43) Pub. Date: Oct. 6, 2016

(54) METHOD AND APPARATUS FOR CARRYING OUT ENDOTHERMIC REACTIONS

(71) Applicant: BASF SE, Ludwigshafen (DE)

(72) Inventors: Kati BACHMANN, Mannheim (DE); Friedrich GLENK, Mannheim (DE); Grigorios KOLIOS, Neustadt (DE)

(73) Assignee: BASF SE, Ludwigshafen (DE)

(21) Appl. No.: 15/038,205

(22) PCT Filed: Nov. 20, 2014
(86) PCT No.: PCT/EP2014/075155

§ 371 Date: May 20, 2016

(30) Foreign Application Priority Data
Nov. 21, 2013 (EP) 13193895.3

(51) Int. Cl. C07C 2/76; C01B 3/38; B01J 7/00; B01J 19/00; B01J 29/48; B01J 23/62; B01J 21/04; B01J 21/06; B01J 23/26; B01J 23/08; B01J 29/18; B01J 29/70; B01J 29/85; B01J 27/232; B01J 23/745; B01J 23/755; B01J 23/02; B01J 23/00; C07C 5/333; C07C 5/03; C07C 5/10; C01B 3/40

(52) U.S. Cl. CPC C07C 2/76; C01B 3/40; C01B 3/384; B01J 7/00; B01J 19/0046; B01J 29/48; B01J 23/626; B01J 21/04; B01J 21/066; B01J 23/26; B01J 23/08; B01J 21/063; B01J 29/18; B01J 29/70; B01J 29/85; B01J 27/232; B01J 23/745; B01J 23/755; B01J 23/02; B01J 23/005; C07C 5/3332; C07C 5/03; C07C 5/10; C01B 2203/0233; C01B 2203/0833; C01B 2203/1241; C01B 2203/141; B01J 2219/00495; B01J 2219/00585; B01J 2219/00594; B01J 2219/0072; B01J 2219/00756; B01J 2219/185; C07C 2529/48; C07C 2529/46; C07C 2521/04; C07C 2521/06; C07C 2523/42; C07C 2523/14; C07C 2523/26; C07C 2529/18; C07C 2523/08; C07C 2523/04

(57) ABSTRACT

The present invention relates to a method for carrying out endothermic reactions comprising the method steps of:

a) externally heating at least two reaction tubes (5), wherein the reaction tubes (5) have been arranged vertically in a heating chamber (3) and each of the reaction tubes (5) has been at least partially packed with a fluidizable material,

b) introducing at least one gaseous reactant (R) into the reaction tubes (5),

c) forming a fluidized bed (7) in the reaction tubes (5),
d) carrying out the endothermic reaction in the reaction tubes (5) at a first temperature (T1) and a first pressure (P1), wherein the reaction volume has been distributed over at least two of the reaction tubes (5), and

e) discharging the reaction product (P) from the reaction tubes (5).

The present invention further relates to an apparatus (1) for carrying out endothermic reactions comprising at least one heating chamber (3), at least two reaction tubes (5), wherein the reaction tubes (5) have been arranged vertically in the heating chamber (3) and each of the reaction tubes (5) comprises an at least partial packing of a fluidizable material, at least one entry point (9) for gaseous reactants (R) for each reaction tube (5), at least one exit point (11) for reaction products (P) for each reaction tube (5) and at least one heating apparatus (13) for externally heating the reaction tubes (5).

The present invention further provides for the use of the apparatus (1) according to the invention for the non-oxidative dehydroaromatization of C₁ to C₄ aliphatics.

The present invention relates to a method and an apparatus for carrying out endothermic reactions, in particular strongly endothermic reactions requiring a large amount of energy.

Endothermic catalytic reactions are often at the top of the chemical industry value chain, for example in the cracking of crude oil fractions, the reforming of natural gas or naphtha, the dehydrogenation of propane or the dehydroaromatization of methane to give benzene. These reactions are strongly endothermic. The energy required for elimination of two hydrogen atoms from an alkane molecule is about 100 kJ/mol to 125 kJ/mol. Temperatures between 500° C. and 1200° C. are necessary to achieve industrially and economically attractive yields. This is mainly due to the thermodynamic limitation of the equilibrium conversion. Providing the necessary heat of reaction at this temperature level is a great technical challenge. The propensity for coking of organic compounds at high temperatures provides a further challenge. The coke is deposited on the catalyst surface and preferentially on the surface of reactor internals, for example on heat transfer surfaces. This deactivates the catalyst and also reduces heat transfer performance. This leads to reduced reactor production capacity. Prior art endothermic heterogeneously catalyzed gas-phase reactions are carried out either in fixed-bed reactors or in fluidized-bed reactors.

In fixed-bed reactors, the necessary process heat is generally provided via a salt melt or flue gases and indirectly transferred from the heat-transfer medium to the catalyst through the tube wall (Ullmann's Encyclopedia of Industrial Chemistry, 7th Edition, Wiley, 2010); Catalytic Fixed-bed reactors, Gerhart Eigenberger, Wilhelm Ruppel). Indirect heat transfer avoids detrimental contamination or dilution of the product stream by the exhaust gases of the combustion. In order to achieve effective temperature control, fixed-bed reactors consist of thin reaction tubes combined to form a tube bundle. The capacity of tube bundle reactors is reliably scaleable since it can be realized via the number of reaction tubes. This construction is attributable to the low radial thermal conductivity of fixed beds of λ_{rad}≦10 W/(m·K), i.e. the transport in fixed beds is limited due to the effective radial coefficient of thermal conductivity. Thus—despite the high slenderness ratio of the reaction tubes—in reactions evolving a large amount of heat distinct radial temperature gradients arise between the tube wall and the tube axis. This can lead to selectivity losses and non-uniform catalyst deactivation. Industrial tube bundle reactors consist of up to 35 000 individual tubes with diameters of between 16 mm and no more than 100 mm. The disadvantage of this is that constructing a tube bundle reactor becomes inconvenient and costly. Not only is the equipment very complex, but it is also very difficult to ensure uniform flow distribution through all of the reaction tubes despite an elaborate procedure for packing the tubes with catalyst.

Particularly for high production capacity processes, fluidized-bed reactors have proven themselves to be the preferred technical concept. Specifically for reactions evolving a large amount of heat, fluidized-bed reactors offer the advantage of high axial and lateral thermal conductivity (λ>100 W/(m·K)) which achieves a homogeneous temperature range in the reaction chamber.

A typically constructed fluidized bed is continuous. The advantage of this construction is that it makes transverse flow equilibration possible. However, this construction also has various disadvantages. For instance, fluidized-bed reactors have a low slenderness or length/diameter ratio (L/D ratio). The L/D ratio is typically in the range between 1 and 3. This results in pronounced axial backmixing, both in the fluidizable material and in the reaction mixture, which generally has a negative effect on reaction yield. Moreover, the reactor wall needs to be very strong in order to ensure mechanical stability especially when operated under pressure.

The prior art discloses various technical solutions for introducing heat to fluidized beds. Heat is generally supplied via immersed tubular coils (cf. “Handbook of Fluidization and Fluid-Particle Systems”, Wen-Ching Yang; Marcel Dekker, Inc., 2003). This concept requires little capital expenditure and—similarly to tube bundle fixed-bed reactors—offers the advantage of indirect heat transfer, namely material separation between reaction gas and heat-transfer medium. This type of reactor is disadvanteagous in that during endothermic reactions high temperatures are generated on the inside of the heat exchanger tubes. Thereby, metallic tube wall are directly exposed to the hot heat transfer medium (fuel gas, exhaust gas). This fact and the requirement that appropriate, and costly, superalloys be used often render a method uneconomical.

Moreover, due to their high slenderness ratio, the heat exchanger tubes are susceptible to resonance oscillations induced by the pulsations of the fluidized bed. The frequency at which a bubble-forming fluidized bed oscillates/pulsates depends primarily on the bubble frequency. This is typically 2 Hz to 14 Hz (cf. Fluidization Engineering, 2nd Edition, Butterworth-Heinemann, 1991; Daizo Kunii, Octave Levenspiel). The eigenfrequency of a commonly used steel heat exchanger tube of length L=10 m and of outer diameter Dₐ=100 mm is about 3 Hz. Since this eigenfrequency of the heat exchanger tubes is of the same order of magnitude as the frequency of the fluidized bed oscillation/fluidized bed pulsation, there is a possibility of resonance and hence of damage to the heat exchanger tubes.

An alternative proposed in the prior art (cf. Fluidization Engineering, 2nd Edition, Butterworth-Heinemann, 1991; Daizo Kunii, Octave Levenspiel) is the use of circulating particle streams, e.g. catalyst particles, for introducing heat. In this technique, the catalyst particles alternately pass through a production cycle and a regeneration cycle in a circulating fluidized bed. The particles thus serve not only as catalyst but also as heat-transfer medium to provide heat for the endothermic reaction. In the reaction chamber, the catalyst particles are cooled down by the endothermicity of the reaction and continuously loaded with carbonaceous deposits (coke). To heat them up and to remove the carbonaceous layer, said particles are treated with a hot regeneration gas in the regeneration zone. However, this technique requires particles resistant to oxygen and mechanical influences, inparticular catalyst particles.

As an alternative, US 2012/0022310 A1 proposes using as heat-transfer medium inert particles meeting the chemical and mechanical requirements. Here, the catalyst particles are operated as an active component of a stationary fluidized bed through which the heated-up inert particles migrate from top to bottom in order to introduce the energy to the fluidized bed. At the lower end of the fluidized bed, the inert particles are discharged, reheated (for example by direct combustion of a fuel) and returned to the fluidized bed from the top of the reaction tube, i.e. from the reactor head. One disadvantage of this method is the mechanical stress which the catalyst particles are subjected to due to collisions with the inert particles and which can lead to catalyst abrasion or even to breakage of the catalyst particles.

For example, the prior art (cf. Ullmann's Encylopedia of Industrial Chemistry, 7th Edition, Wiley, 2010; Benzene; Hillis O. Folkins) discloses carrying out the dehydroaromatization of methane in a fluidized-bed reactor using a pulverulent catalyst as the fluidizable material. A reaction temperature in excess of 520° C. is required. Here, an alkane is supplied to the lower end of the reaction tube of the fluidized-bed reactor and converted into benzene and further hydrocarbons as by-products in the reaction space (in the fluidized bed). The energy required for the reaction ideally needs to be supplied to the system directly, in order to avoid loss of selectivity by uncontrolled reactions on superheated surfaces.

US 2007/0249880 A1 describes the production of aromatics from methane. Here, the dehydroaromatization is carried out in a fluidized bed of catalyst material which in addition to its character as fluidizable material also serves as heat-transfer material by circulating between production and regeneration steps. US 2008/0249343 A1 proposes a similar technology.

Disadvantages of the known prior art consequently include high capital expenditure and the complexity of the reactors (in particular for tube bundle reactors) and also the limited use potential for fluidized-bed reactors on account of the limitations imposed by the fluidizable material (catalyst) and/or the heat-transfer medium. In particular, scaling-up fluidized-bed reactors is not straightforward.

It is thus an object of the present invention to provide an improved method for carrying out endothermic reactions and an improved apparatus for carrying out endothermic reactions which can be used to overcome the disadvantages of the prior art. The objective is in particular to be able to carry out endothermic reactions with acceptable capital expenditure and with ideally optimal resource utilization.

The object is achieved by a method for carrying out endothermic reactions comprising the method steps of:
a) externally heating at least two reaction tubes (5), wherein the reaction tubes (5) have been arranged vertically in at least one heating chamber (3) and each of the reaction tubes (5) has been at least partially packed with a fluidizable material,
b) introducing at least one gaseous reactant (E) into the reaction tubes (5),
c) forming a fluidized bed (7) in the reaction tubes (5),
d) carrying out the endothermic reaction in the reaction tubes (5) at a first temperature (T1) and a first pressure (P1), wherein the reaction volume has been distributed over at least two of the reaction tubes (5), and
e) discharging the reaction product (P) from the reaction tubes (5).

The method according to the invention can be carried out using the apparatus (1) according to the invention. The apparatus (1) according to the invention for carrying out endothermic reactions comprises
at least one heating chamber (3),
at least two reaction tubes (5), wherein the reaction tubes (5) have been arranged vertically in the heating chamber (3) and each of the reaction tubes (5) comprises an at least partial packing of a fluidizable material,
at least one entry point (9) for gaseous reactants (E) for each reaction tube (5),
at least one exit point (11) for reaction products (P) for each reaction tube (5) and
at least one heating apparatus (13) for externally heating the reaction tubes (5).

The method according to the invention combines the advantages of a reaction in a fluidized bed and of a reaction in a tube bundle reactor, i.e., indirect heating of the catalyst material is realized by indirect heating of a plurality of fluidized beds disposed in individual reaction tubes. The reaction volume here need not be continuous but rather can be distributed over a plurality of reaction tubes installed vertically in a combustion chamber. Supplying the heat of reaction via indirect heating through the walls of the reaction tubes (5) together with the high heat transfer coefficient (heat transfer from the fluidized bed to the tube wall) offered by a fluidized bed (α˜100 W/(m²·K) to 1000 W/(m²·K)) makes it possible to achieve a virtually isothermal reaction zone distributed over the reaction tubes. This considerably simplifies the method procedure and simultaneously reduces costs compared to prior art methods.

A further advantage of the present invention is the reduced particle and gas backmixing on account of a high L/D ratio between the length L of the fluidized bed and the diameter D thereof (also L/D ratio or slenderness ratio) of about 3 to 30 compared to conventional fluidized beds having an L/D ratio of from 1 to 3. This makes it possible to achieve higher selectivities and improved yields.

The apparatus (1) according to the invention exhibits distinctly improved heat transfer compared to conventional fixed-bed reactors (tube bundle fixed bed reactors). The construction of the apparatus (1) according to the invention exhibits reduced equipment complexity compared to a fluidized-bed reactor which uses inert particles as heat-transfer medium since it is not necessary to provide a particle system for circulating the inert particles. This also reduces the mechanical abrasion on the catalyst particles arising on account of circulation through inert particles. Moreover, the space-time yield of the reactor rises as no inert particles block a part of the reaction volume. Finally, the process procedure is distinctly simplified as it is no longer necessary to handle inert particles.

A further significant advantage compared to conventional tube bundle reactors is that the individual reaction tubes (5) can have a much larger diameter (up to 1500 mm, in some cases up to 3000 mm). The number of tubes is thus reduced considerably, thereby simplifying the reactor construction. It is moreover simpler to ensure equal distribution of the flow through the reaction tubes (5) by packing all tubes of the apparatus (1) with the same catalyst mass.

Internal heat exchanger surfaces, i.e. fittings within the reaction tubes, are unnecessary in the apparatus (1) according to the invention. The fluidizable material thus moves in a direction substantially parallel to the walls of the reaction tubes (5). This is particularly advantageous for two reasons:
1. The susceptibility to abrasion of the reaction tubes (5) is reduced considerably.
2. In reactions having a propensity for depositing carbonaceous material (coking), the formation of deposits on the walls of the reaction tubes (5) and the consequent blockage of the flow cross section is suppressed.

Furthermore, the load on the materials of construction is reduced in the apparatus (1) according to the invention since the large diameter of the reaction tubes (5) eliminates the risk of resonance oscillations initiated by the pulsation of the fluidized bed. The eigenfrequency of the materials used is therefore significantly higher than the pulsation frequency of the fluidized bed. For example, the eigenfrequency of a tube of length L=10 m and of outer diameter D=1000 mm is about 26 Hz. Thus, in the apparatus (1) according to the invention the risk of such oscillations (i.e. resonance oscillations) leading to stresses in the material of construction and ultimately to acceleration of any cracks that arise, damaging the mucrostructure of the tube wall, is distinctly minimized.

The invention is described in more detail hereinbelow.

The present invention firstly provides (as already specified hereinabove) a method for carrying out endothermic reactions comprising the method steps a) to e). The method according to the invention is preferably carried out using the apparatus (1) according to the invention (likewise specified hereinabove). If in connection with the method according to the invention the text which follows also specifies apparatus features, such apparatus features preferably relate to the apparatus (1) according to the invention which is more particularly defined in connection with the method according to the invention.

In the context of the present invention, the term “endothermic reactions” is generally understood as meaning reactions having a reaction enthalpy (−ΔHᵣ)<0 (cf. Ullmann's Encylopedia of Industrial Chemistry, 7th Edition, Wiley, 2010; Principles of Chemical Reaction Engineering, K. Roel Westerterp, Ruud J. Wijngaarden). Such reactions can be elimination reactions, dehydrogenations, dehydrations, hydrocarbon cracking reactions, decomposition reactions, carbon-carbon coupling reactions of hydrocarbons or combinations thereof.

Method step a) comprises externally heating at least two reaction tubes (5), wherein the reaction tubes (5) have been arranged vertically in at least one heating chamber (3) and each of the reaction tubes (5) has been at least partially packed with a fluidizable material. The externally heating in particular is an indirect heating.

The term “heating chamber” is understood to mean an essentially sealed space into which energy is introduced in various ways, said energy being transferred to the reaction tubes (5) arranged in the heating chamber (3). The purpose of the heating chamber (3) according to the invention is, in particular, to ensure uniform heating of the reaction tubes (5). In the present case, “uniform” means that the variance in the distribution of the heat flow density over the circumference of the reaction tubes (5) should not exceed 30% and preferably should not exceed 15% and that the variance from reaction tube to reaction tube of the heat flow must not exceed 30% and preferably must not exceed 15%.

A temperature variation of 100 K is detrimental to dehydrogenation processes for example. When there is too great a decrease in temperature the reactants cease to react and when there is too great an increase in temperature the selectivity for the carbonaceous deposits (coke) also increases so reducing the yield of the target products. This is shown below in the embodiments.

There are at least two reaction tubes (5). In the method according to the invention preference is given to using 2 to 15 000 tubes ins particular 10 to 10 000 tubes, preferably 20 to 10 000 tubes, particularly 50 to 5000 tubes and more preferably 100 to 5000 tubes.

In accordance with the invention it is possible to use as fluidizable material particles from the classification groupings Geldart A and/or Geldart B and/or Geldart C and/or Geldart D and mixtures thereof, said groupings being known to one skilled in the art. Geldart A comprises particles having a low mean particle size and a density of less than 1.4 g/cm³, Geldart B comprises particles having a size of from 40 μm to 500 μm and a density between 1.4 g/cm³ and 4.0 g/cm³ , Geldart C comprises particles having a size of from 20 μm to 30 μm, Geldart D comprises particles having a size of >500 μm and a density between 1.4 g/cm³ and 4.0 g/cm³ (cf. “Types of Gas Fluidization”, D. Geldart, Powder Technology, 7 (1973) 285-292).

At least 50% of the particles preferably comprise at least one component which is active for the reaction according to the invention.

The dehydroaromatization of methane to give benzene can be carried out using, for example, catalysts comprising a porous support having at least one metal applied thereto. It is preferable in accordance with the invention when the support comprises at least one zeolite, it is particularly preferable when the support has a structure selected from the structure types pentasil and MWW and it is especially preferable when the support has a structure selected from the structure types MFI, MEL and mixed MFI/MEL and MWW structure types. It is very particularly preferable to use a zeolite of the type ZSM-5 or MCM-22. The descriptions of the structure types of the zeolites correspond to those of W. M. Meier, D. H. Olson and Ch. Baerlocher (cf. “Atlas of Zeolite Structure Types”, Elsevier, 3^{rd} edition, Amsterdam 2001). These zeolite particles can be classifed into the group Geldart A.

The catalyst, e.g. for the dehydroaromatization, typically comprises at least one metal selected from groups 3 to 12 of the periodic table. It is preferable in accordance with the invention when the catalyst comprises at least one element selected from the transition metals of main groups 6 to 11. It is particularly preferable when the catalyst comprises Mo, W, Re, Fe, Ru, Os, Co, Rh, Ir, Ni, Pd, Pt, Cu. It is very particularly preferable when the catalyst comprises at least one element selected from the group Mo, W and Re. It is likewise preferable in accordance with the invention when the catalyst comprises at least one metal as active component and at least one further metal as dopant. In accordance with the invention the active component is selected from Mo, W, Re, Ru, Os, Rh, Ir, Pd, Pt. In accordance with the invention the dopant is selected from the group Cr, Mn, Fe, Co, Ni, C, V, Zn, Zr and Ga, preferably from the group Fe, Co, Ni, Cu. In accordance with the invention the catalyst can comprise more than one metal as active component and more than one metal as dopant. These are each selected from the metals listed for the active component and the dopant.

Moreover, non-metallic catalysts can be applied for other reaction systems.

It has proved advantageous for the efficiency of the method according to the invention when the endothermic reaction is heterogeneously catalyzed and the fluidizable material is a fluidizable catalyst useful for the endothermic reaction. In contrast to the prior art methods the catalysts of the present invention are not exposed to the flue gases of the combustion used for heat generation and, as a result, said catalysts need not necessarily be chemically and mechanically stable toward such conditions. This broadens the range of industrially usable catalysts.

Method step b) comprises introducing at least one gaseous reactant (E) into the reaction tubes (5). A useful gaseous reactant is selected depending on the specific endothermic reaction to be carried out. The range of appropriate reactants is known to one skilled in the art. Examples include: CH₄ for the dehydroaromatization of methane to give benzene, C₃H₈, H₂O and H₂ for the dehydrogenation of propane to give propylene, C₄H₁₀, H₂O and H₂ for the dehydrogenation of butane to give butene, C₈H₁₀ and H₂O for styrene synthesis, CH₄ and H₂O for steam reforming and CH₄ and CO₂ for dry reforming of natural gas to give synthesis gas, CH₄ for natural gas pyrolysis. Beside the reactant, impuriteies are contained in the raw material, which may be chemically inert or chemically active. The chemically inert materials leave the reactor unchanged, while the chemically active components are converted completely or partially in the reactor.

In accordance with the invention, method step c) comprises forming a fluidized bed (7) in the reaction tubes (5). The fluidized bed (7) can be operated both in the bubble-forming and turbulent regime or in the “fast fluidization” regime. The regimes are classified according to the Grace diagram known to one skilled in the art (cf. Fluidization Engineering, 2nd Edition, Butterworth-Heinemann, 1991; Daizo Kunii, Octave Levenspiel).

Method step d) comprises carrying out the endothermic reaction in the reaction tubes (5) at a first temperature (T1) and a first pressure (P1), wherein the reaction volume has been distributed over at least two of the reaction tubes (5). The first temperature (T1) chosen in method step d) and the first pressure (P1) depend primarily on the endothermic reaction to be carried out. The temperature and pressure ranges useful for particular reactions are known to one skilled in the art. It is preferable when the temperature (T1) is 500° C. to 1000° C., preferably 500° C. to 900° C., more preferably 600° C. to 850° C. The first pressure (P1) is 0.1 bar to 30 bar, preferably 0.1 bar to 20 bar, more preferably 0.1 bar to 10 bar. The pressure (P1) is in particular the absolute pressure.

Method step e) comprises discharging the reaction product (P) from the reaction tubes (5). The specific reaction products (P), i.e. the composition of the reaction product, is/are known to one skilled in the art and consists of volatile, inder reaction conditions gaseous substances, which are formed depending on the specific endothermic reaction carried out. The reaction products (P) can be a single product or two or more products. The reaction product likewise comprises by-products and/or impurities.

Since carbonaceous material (coke) can be deposited on the catalyst during the method according to the invention, the method according to the invention preferably comprises method step f) regenerating the catalyst at a second temperature (T2) and a second pressure (P2) using a suitable regeneration gas (R).

The conditions suitable for regenerating the catalyst material, i.e., for removing the carbonaceous deposits on the catalyst particles, such as the second temperature (T2), the second pressure (P2) and the feed composition generally differ from the temperatures and pressures required for the endothermic reaction (T1 and P1) and the feed compositions required therefor. It is therefore advantageous to provide a separate method step for regenerating the catalyst.

The feed composition is the composition of the fluid stream introduced into the reaction tubes in method step b) and/or f).

It is preferable when the temperature (T2) is 500° C. to 1000° C., preferably 500° C. to 900° C., more preferably 600° C. to 850° C. The second pressure (P2) is 0.1 bar to 30 bar, preferably 0.1 bar to 20 bar, more preferably 0.1 bar to 10 bar. In particular, this applies for the dehydroaromatization.

Although the stated ranges for the temperatures (T1, T2) and the pressures (P1, P2) appear not to differ, the actual temperatures (T1, T2) and pressures (P1, P2) can be adjusted differently depending on the specific methods. In the case of dehydroaromatization for example, the endothermic reaction is carried out in particular at low pressure while the regeneration is particularly effective at high pressure.

In particular, method step f) can be carried out wholly or partially in parallel with method steps b), c), d) and e) and the endothermic reaction therefore need not be interrupted at any time. In this connection it is additionally advantageous when the number of reaction tubes (5) in production mode is variable and one or more reaction tubes (5) can be brought on- or offline according to demand for the endothermic reaction. In this connection “variable” means that—depending on the required reaction volume—one or more reaction tubes (5) are used for the endothermic reaction while the remaining reaction tubes (5) are used for the regeneration or are idle.

In one development the reaction tubes (5) can be combined to form groups which independently of one another are alternately operated in a production mode and/or in a regeneration mode or are idle.

In accordance with the present invention “production mode” is understood as meaning a process step comprising one or more of the reaction types, wherein these reaction types comprise, for example, an elimination reaction, dehydrogenation, hydrocarbon cracking, dehydration, aromatization or decomposition reactions.

In accordance with the present invention “regeneration mode” is understood as meaning a process step comprising one or more of the following steps: purging with inert gas, oxidation of one or more components of the catalyst with lean air or with undiluted air, reduction of one or more components of the catalyst, gasification of carbonaceous deposits on the catalyst with, for example, CO₂, H₂ or H₂O.

In accordance with the present invention “idle” is understood as meaning a state in which one or more reaction tubes (5) or reaction tubes (5) combined to form groups are operated neither in production mode nor in regeneration mode.

The variable operation of individual reaction tubes (5) or reaction tubes (5) combined to form groups makes it possible to configure the throughput of the method according to the invention according to demand without additional capital expenditure and without significantly altering the reaction procedure. It is further possible to switch a number of reaction tubes (5) over to a regeneration cycle while other reaction tubes (5) are run in the production cycle. This means that an endothermic reaction need not be stopped in order to regenerate the catalyst material but rather it can be carried out as a substantially continuous operation. In addition individual reaction tubes (5) or reaction tubes (5) combined to form groups can be idle when said tubes are not needed for the capacity required at a particular juncture.

In one development of the method according to the invention the gaseous reactant (E) and the regeneration gas (R) are introduced into the respective reaction tubes (5) at at least two different points. Said gases are preferably introduced simultaneously. Here, the fluidized bed (7) is a fluidized bed vertically divided into zones and having a production zone and a regeneration zone between which the catalyst particles periodically circulate. This reduces the mechanical stress due to pressure and temperature variations over time.

Since the method according to the invention is intended for carrying out strongly endothermic reactions, method step a) comprises introducing at least 5 MW and in particular between 50 MW and 500 MW of power.

The method according to the invention is used in particular for the non-oxidative dehydroaromatization of C₁ to C₄ aliphatics since the energy requirements of this endothermic reaction are particularly great.

The non-oxidative dehydroaromatization of C₁ to C₄ aliphatics is preferably carried out using a catalyst comprising a porous support having at least one metal applied thereto. It is preferable in accordance with the invention when the support comprises at least one zeolite, it is particularly preferable when the support has a structure selected from the structure types pentasil and MWW and it is especially preferable when the support has a structure selected from the structure types MFI, MEL and mixed MFI/MEL and MWW structure types. It is very particularly preferable to use a zeolite of the type ZSM-5 or MCM-22. The descriptions of the structure types of the zeolites correspond to those of W. M. Meier, D. H. Olson and Ch. Baerlocher (cf. “Atlas of Zeolite Structure Types”, Elsevier, 3^{rd} edition, Amsterdam 2001). These zeolite particles can be classifed into the group Geldart A.

The catalyst typically comprises at least one metal selected from groups 3 to 12 of the periodic table. It is preferable in accordance with the invention when the catalyst comprises at least one element selected from the transition metals of main groups 6 to 11. It is particularly preferable when the catalyst comprises Mo, W, Re, Fe, Ru, Os, Co, Rh, Ir, Ni, Pd, Pt, Cu. It is very particularly preferable when the catalyst comprises at least one element selected from the group Wo, W, and Re. It is likewise preferable in accordance with the invention when the catalyst comprises at least one metal as active component and at least one further metal as dopant. In accordance with the invention the active component is selected from Mo, W, Re, Ru, Os, Rh, Ir, Pd, Pt. In accordance with the invention the dopant is selected from the group Cr, Mn, Fe, Co, Ni, C, V, Zn, Zr and Ga, preferably from the group Fe, Co, Ni, Cu. In accordance with the invention the catalyst can comprise more than one metal as active component and more than one metal as dopant. These are each selected from the metals listed for the active component and the dopant.

For the abovementioned non-oxidative dehydroaromatization the first temperature (T1) is 600° C. to 800° C., the second temperature (T2) is 500° C. to 800° C., the first pressure (P1) is 0.1 bar to 10 bar and the second pressure (P2) is 0.1 bar to 30 bar. The pressures (P1, P2) are in particular absolute pressures.

The present invention further provides (as specified above) the apparatus (1) for carrying out endothermic reactions comprising
at least one heating chamber (3),
at least two reaction tubes (5), wherein the reaction tubes (5) have been arranged vertically in the heating chamber (3) and each of the reaction tubes (5) comprises an at least partial packing of fluidizable material,
at least one entry point (9) for gaseous reactants (E) for each reaction tube (5),
at least one exit point (11) for reaction products (P) for each reaction tube (5) and
at least one heating apparatus (13) for externally heating the reaction tubes (5).

The apparatus (1) according to the invention is preferably used in the method described hereinabove for carrying out endothermic reactions. If method features are described in connection with the apparatus (1) in the text which follows, reference is made, unless stated otherwise, to the corresponding indications as in the method according to the invention described hereinabove.

The apparatus (1) is advantageously of a modular construction and therefore at least two reaction tubes (5) can be brought on- or offline for the endothermic reaction. This distinctly enhances the flexibility of the apparatus (1) according to the invention. As already explained in connection with the method, the throughput of gaseous reactants (E) can be adjusted according to demand by bringing on- or offline individual reaction tubes (5) or reaction tubes (5) combined to form groups. In this way, an endothermic reaction optimized on a relatively small scale can be readily replicated as a relatively high-throughput reaction. While conventional fluidized-bed reactors require costly and inconvenient “scaling up”, “numbering up” is sufficient in the present case since what is involved here is merely combining with one another a plurality of reaction tubes (5) which comprise a fluidized bed (7) and which have been optimized in terms of their throughput and for sufficient heat introduction. It is thus possible to vary the size of the plant and hence the throughput of the reaction within wide limits. The apparatus according to the invention consequently has an extremely wide load range.

When reversible deactivation occurs the catalyst can be regenerated in the apparatus (1) according to the invention. To this end, the apparatus (1) can be divided into segments which can be switched between production mode and regeneration mode independently of one another. Dividing the reaction volume over a plurality of reaction tubes (5) offers the advantage that some of these reaction tubes (5) are operated in regeneration mode while the remaining reaction tubes (5) are run in production mode. This makes it possible to regenerate the catalyst in periodic time intervals without interrupting production.

While conventional prior art fixed-bed reactors often comprise reaction tubes of up to 100 mm in diameter, each of the reaction tubes (5) in the apparatus (1) according to the invention preferably has a diameter of more than 100 mm, in particular a diameter of from 125 mm to 1500 mm, ins some cases up to 3000 mm. This drastically reduces the number of tubes required in the apparatus (1) according to the invention. For a dehydroaromatization for example, an apparatus (1) according to the invention requires the use of about 3000 tubes given a tube diameter of 500 mm while for the same capacity and under identical operating conditions a tube bundle fixed-bed reactor with tubes of no more than 100 mm in diameter would require the use of about 75 000 tubes. Operating data used as a basis for this calculation were a gas entry temperature of 550° C., a reaction temperature of 700 ° C. and an absolute operating pressure of 4 bar. Here, the required amount of heat of reaction at 8% methane to benzene conversion was almost 140 MW. The total gas flow is around 960 t/h of CH₄.

In order to be able to carry out the endothermic reactions optimally it has proven advantageous when the heating apparatus (13) of the apparatus (1) according to the invention has been configured to provide heat output of at least 5 MW, in particular between 50 MW and 500 MW.

Another development of the apparatus (1) according to the invention provides that at least two reaction tubes (5) are connected to one another. This connection is effected in particular at the inlets and/or the outlets of the reaction tubes (5). This achieves the principle of communicating pipes and the levels of the fluidized beds in all reaction tubes (5) connected to one another therefore substantially equilibrate. Equal distribution is thus ensured independently of initial packing. This development moreover makes it possible to achieve simpler, faster and thus more efficient packing of the plant.

The present invention further provides for the use of the apparatus (1) described hereinabove for the non-oxidative dehydroaromatization of C₁ to C₄ aliphatics. Non-oxidative dehydroaromatizations of C₁ to C₄ aliphatics as such are (as already noted hereinabove) known to one skilled in the art.

Strongly endothermic reactions such as the non-oxidative dehydroaromatizations of C₁ to C₄ aliphatics can no longer be carried out economically on an ever larger scale with conventional heat exchangers in conventional tube bundle reactors or fluidized-bed reactors. The use of the apparatus (1) according to the invention for the non-oxidative dehydroaromatization of C₁ to C₄ aliphatics therefore offers distinct economic advantages.

The apparatus (1) according to the invention is described hereinbelow as a “tube bundle fluidized-bed reactor”.

Further objectives, features, advantages and possible applications will become apparent from the following description of the working examples of the present invention with reference to the figures. All features described and/or illustrated in figures, alone or in any combination, form the subject matter of the present invention irrespective of their combination in the claims or the claims to which they refer back.

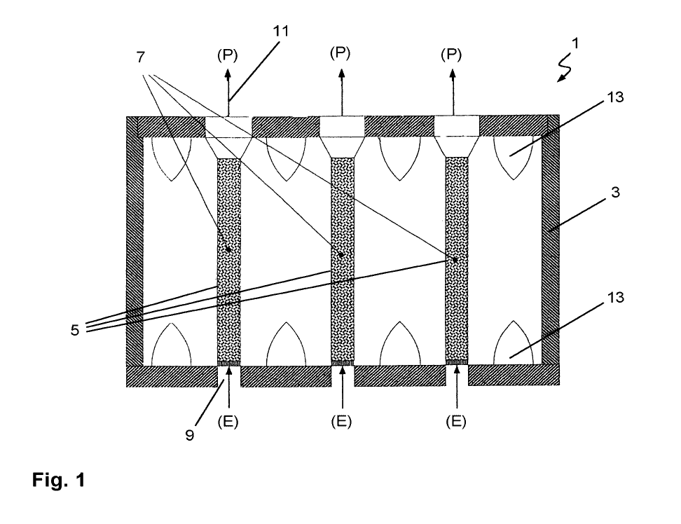

FIG. 1 shows a schematic diagram of a tube bundle fluidized-bed reactor (1) in one embodiment of the invention and

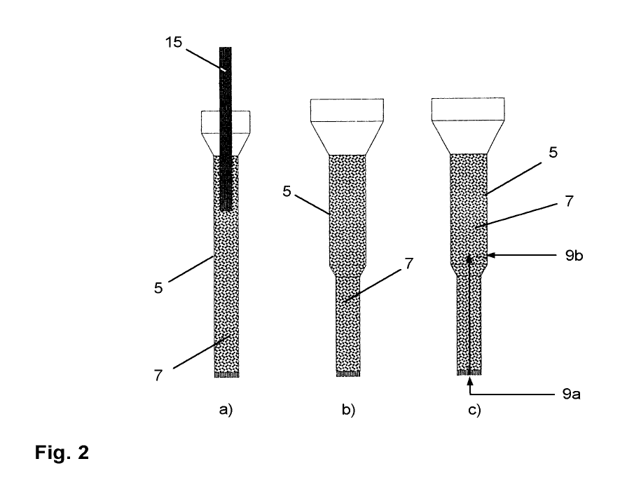

FIG. 2 shows schematic diagrams a), b) and c) of three different embodiments of the reaction tubes (5) according to the present invention.

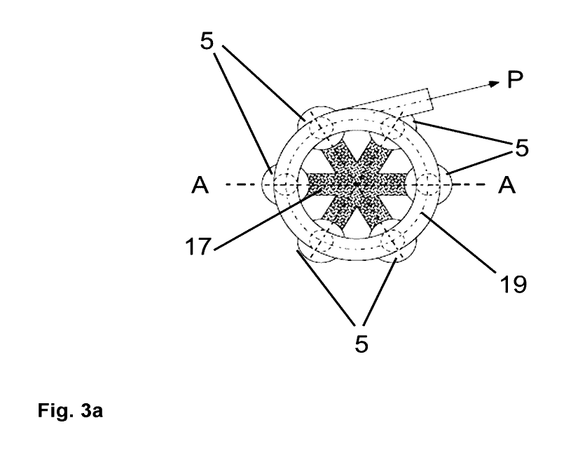

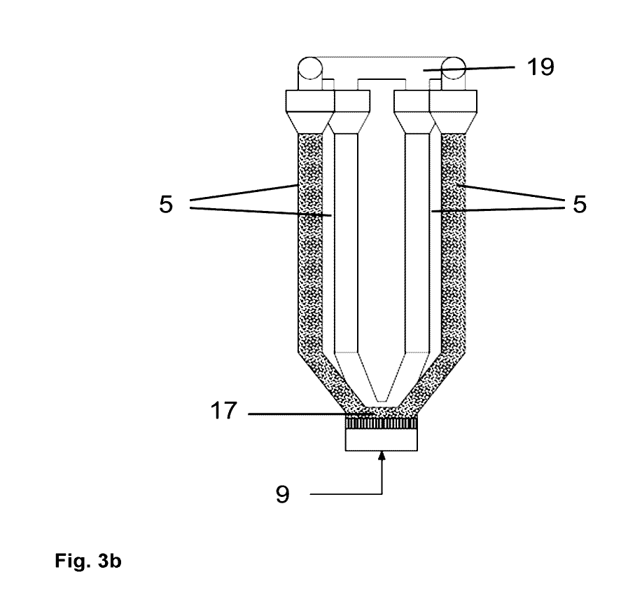

FIG. 3a shows a schematic diagram of a group of reaction tubes in plan view which are connected to one another via a common inlet and a common outlet and

FIG. 3b shows a schematic sectional diagram along the line A-A of the group of reaction tubes in FIG. 3a.

FIG. 1 shows a schematic diagram of a tube bundle fluidized-bed reactor 1 according to the invention for endothermic high temperature reactions. The reaction tubes 5 are arranged vertically in the combustion chamber 3. The reaction tubes 5 comprise fluidizable material in order to form a fluidized bed 7. In a preferred embodiment, the reactant stream E is introduced into the reaction tube 5 from below via entry point 9 to fluidize the fluidizable material to form a fluidized bed 7 and also to be converted into product P in the endothermic reaction. The product stream P is withdrawn at the top of the reaction tubes 5 via exit points 11.

In the embodiment shown in FIG. 1, the combustion chamber 3 is fired via jet burners as heating apparatuses 13. The jet burners 13 can be fueled with natural gas, retentate streams from separation steps, offgases from purification steps or fuel-like products from other processes for example.

The configuration shown in FIG. 1, when the heating apparatuses 13 are directed into the combustion chamber 3 from both above and below, makes it possible to realize different temperatures over the length of the reaction tubes 5, in particular a temperature gradient.

The FIGS. 2a, 2b and 2c show three embodiments of the reaction tubes 5.

FIG. 2a shows an immersed tube 15 in the reaction tube 5 through which catalyst particles can be supplied and/or withdrawn during operation. This makes it possible to compensate the catalyst mass loss due to abrasion in the fluidized bed 7 for example. Moreover, catalyst particles can be withdrawn in order to change the volume of the fluidized bed 7 or to regenerate the catalyst material externally. It is also simpler to change the catalyst because in the present embodiment catalyst can be continuously withdrawn and replaced with fresh catalyst during operation, while in a fixed-bed reactor for example changing the catalyst necessitates shutting down, cooling down and opening the reactor. The present embodiment distinctly reduces downtime and distinctly increases the availability of the reactor. Catalyst changes typically take place every two years.

FIG. 2b shows a reaction tube 5 with a cross section varying over its length. This configuration makes it possible to keep the fluidization regime virtually constant for a reaction with an increase in volume.

FIG. 2c shows a reaction tube 5 having two entry points 9a and 9b by means of which the fluidized bed 7 can be divided into two zones. This raises the possibility of establishing both a reaction zone and regeneration zone in one and the same reaction tube 5. In this case a regeneration gas R is introduced via entry point 9a in order to regenerate the catalyst particles which have been deactivated by carbonaceous deposits (coked). Transport of the particles between the two zones takes place on account of the natural movement of said particles in a fluidized bed. The gaseous reactant E is supplied via entry point 9b.

In FIGS. 2b and 2c, two zones can be formed in the fluidized bed 7 by selecting suitable tube cross sections and by targeted adjustment of the flow rates. When a regeneration zone is formed in the lower region and a reaction zone is formed in the upper region, the catalyst particles can advantageously be regenerated here in a continuous operation during the reaction.

FIG. 3a shows a schematic diagram of a group of reaction tubes 5 in plan view. The reaction tubes 5 are connected to one another via a common inlet 17 and a common outlet 19. This achieves the principle of communicating pipes. The group shown forms one unit of a modular reactor.

FIG. 3b shows a schematic sectional diagram along the line A-A from FIG. 3a. The interconnection of the inlets and the outlets ensures a uniform degree of packing with catalyst in all reaction tubes 5 of the group, i.e., a uniform level of the fluidized beds 7.

Given below are specific working examples for endothermic reactions which may be carried out with the method according to the invention and the apparatus 1 according to the invention.

Dehydroaromatization Reaction and Regeneration of a Catalyst

The dehydroaromatization reaction and the regeneration of a catalyst were carried out in a reactor under the conditions shown in table 1. The WHSV (weight hourly space velocity) is given by the mass flow of methane (for the reaction) or hydrogen (for the regeneration) divided by the amount of catalyst in the plant.

The catalyst employed was a spray-dried ZSM-5 comprising 6% molybdenum and 1% nickel. The particle size was in the range of from 45 μm to 200 μm.

The reaction proceeded at 750° C. and 2.5 bar absolute. This converted 5% of the methane. The benzene selectivity was 80%.

The catalyst was regenerated after a reaction time of 10 h. The regeneration was effected using hydrogen at 810° C. and 4 bar absolute. The hydrogen conversion was 5% and only methane was formed.

Both reactions were carried out in the weakly bubble-forming fluidization state.

TABLE 1

| | Reaction | Regeneration |
|---|---|---|
| T [° C.] | 750 | 810 |
| p [bar gauge] | 1.5 | 3 |
| WHSV [kg/kg/h] | 0.13 | 0.071 |
| X CH₄ or H₂ [%] | 5 (CH₄) | 5 (H₂) |
| S C₆H₆ [%] | 80 | |

Propane Dehydrogenation

Stoichiometric Equation

C₃H₈⇄C₃H₆+H₂(ΔH_{R}=124.25 kj/mol (I)

Catalysts:

Pt/Sn (also other group VIII metals) on Al₂O₃ or ZrO₂

Cr₂O₃ on Al₂O₃ or ZrO₂

Ga₂O₃ on Zeolite (Mordenite, MCM-41, SAPO), TiO₂ or Al₂O₃

Production Phase

Feed Composition

C₃H₈(30-100 vol %), H₂(0-50 vol %), H₂O(0-50 vol %), C₂H₆(<5 vol %), CH₄(<5 vol %) (II)

Operating conditions: temperature: 500-650° C., pressure: 0.3-5 bar_{abs}

Regeneration Phase

Feed Composition

O₂(0-30 vol %), H₂(0-100 vol %, H₂O(0-100 vol %), N₂(0-100 vol %) (II)

Operating conditions: temperature: 500-700° C., pressure: 0.3-5 bar_{abs}

Butane Gydrogenation

Stoichiometric Equation

C₄H₁₀⇄C₄H₈+H₂(ΔH_{R}=125 kj/mol) (IV.1)

C₄H₈⇄C₄H₆+H₂(ΔH_{R}=109.4 kj/mol) (IV.2)

C₄H₁₀: n-butane or isobutane

C₄H₈: 1-butene or isobutene

Catalysts for equation (IV.1):

Pt/Sn (also other group VIII metals) on Al₂O₃ or ZrO₂

Cr₂O₃ on Al₂O₃ or ZrO₂

Ga₂O₃ on Zeolite (Mordenite, MCM-41, SAPO), TiO₂ or Al₂O₃

Catalysts for equations (IV.1) and (IV.2):

Cr₂O₃ on Al₂O₃ or ZrO₂

Production Phase

Feed Composition

C₄H₁₀(30-100 vol %), H₂(0-50 vol %), H₂O(0-50 vol %), C₂H₆(<5 vol %), CH₄(<5 vol %) (V)

Operating conditions: temperature: 500-650° C., pressure: 0.3-5 bar_{abs}

Regeneration Phase

Feed Composition

O₂(0-30 vol %), H₂(0-100 vol %), H₂O(0-100 vol %), N₂(0-100 vol %) (VI)

Operating conditions: temperature: 500-700° C., pressure: 0.3-5 bar_{abs}

Ethylbenzene Hydrogenation

Stoichiometric Equation

C₈H₁₀⇄C₈H₈+H₂(ΔH_{R}(600° C.)=124.9 kj/mol) (VII)

Catalysts

Fe₂O₃/Cr₂O₃/K₂CO₃

Production Phase

Feed Composition

C₈H₁₀(10-50 vol %), H₂(0-10 vol%), H₂O(50-90 vol %), C₆H₆(<5 vol %), C₇H₈(<5 vol %) (VIII)

Operating conditions: temperature: 550-650° C., pressure: 0.3-2 bar_{abs}

Regeneration Phase (Rarely Used)

Feed Composition

O₂(0-30 vol %), H₂(0-100 vol %), H₂O(0-100 vol %), N₂(0-100 vol %) (IX)

Operating conditions: temperature: 500-700° C., pressure: 0.3-5 bar_{abs}

Hydrocarbon Reforming (Natural Gas, Naphtha)

Stoichiometric Equation

$$\begin{matrix}{C}_{n} {H}_{m} + n {H}_{2} O ⇆ n CO + ( n + \frac{m}{2} ) {H}_{2} ( Δ {H}_{R} \approx 206 \cdot n \frac{kJ}{mol} ) & ( X . 1 ) \\ {C}_{n} {H}_{m} + n {H}_{2} O ⇆ n CO + ( n + \frac{m}{2} ) {H}_{2} ( Δ {H}_{R} \approx 206 \cdot n \frac{kJ}{mol} ) & ( X . 2 )\end{matrix}$$

Catalysts

Ni on α-Al₂O₃, MgO or Al—Mg spinel

Ni, Co hexaaluminate

Production Phase

Feed Composition

H₂O₂CₙHₘ(n14n), CO₂: CₙHₘ(0: 2n) (XI)

Operating conditions: temperature: 700-1000° C., pressure: 5-50 bar_{abs}

Regeneration Phase (Rarely Used)

Feed Composition

O₂(0-30 vol %), H₂(0-100 vol %), H₂O(0-100 vol %), N₂(0-100 vol %) (XII)

Operating conditions: temperature: 500-1000° C., pressure: 1-50 bar_{abs}

1. A method for carrying out an endothermic reaction, comprising:
a) externally heating at least two reaction tubes, wherein the reaction tubes are arranged vertically in a heating chamber, and each of the reaction tubes is at least partially packed with a fluidizable material,
b) introducing at least one gaseous reactant into the reaction tubes,
c) forming a fluidized bed in the reaction tubes, each fluidized bed having an L/D ratio between the length L of the fluidized bed and the diameter D thereof from 3 to 30,
d) carrying out an endothermic reaction in the reaction tubes at a first temperature (T1) and a first pressure (P1), wherein the reaction volume is distributed over at least two of the reaction tubes, to obtain a reaction product, and
e) discharging the reaction product from the reaction tubes,
wherein the reaction tubes can be combined to form groups which independently of one another are alternately operated in a production mode and/or in a regeneration mode or are idle.

2. The method according to claim 1, wherein the endothermic reaction is heterogeneously catalyzed and the fluidizable material is a fluidizable catalyst useful for the endothermic reaction.

3. The method according to claim 2, further comprising:
f) regenerating the catalyst at a second temperature (T2) and a second pressure (P2) using a suitable regeneration gas (R).

4. The method according to claim 3, wherein f) is carried out wholly or partially in parallel with b), c), d) and e).

5. The method according to claim 1, wherein the number of reaction tubes in production mode is variable and one or more reaction tubes are brought on- or offline according to demand for the endothermic reaction.

6. The method according to claim 1, wherein the gaseous reactant and the regeneration gas are introduced into the respective reaction tubes at at least two different points.

7. The method according to claim 1, wherein a) comprises introducing at least 5 MW of power.

8. The method according to claim 1, wherein the endothermic reaction is a non-oxidative dehydroaromatization of one or more C₁ to C₄ aliphatics.

9. The method according to claim 8, wherein a catalyst for the non-oxidative dehydroaromatization of the C₁ to C₄ aliphatics is a catalyst comprising a porous support having at least one metal applied thereto.

10. The method according to claim 3, wherein the first temperature (T1) is 500° C. to 1000° C., the second temperature (T2) is 500° C. to 900° C., the first pressure (P1) is 0.1 bar to 10 bar and/or the second pressure (P2) is 0.1 bar to 30 bar.

11. An apparatus suitable for carrying out endothermic reactions comprising
at least one heating chamber,
at least two reaction tubes, wherein the reaction tubes are arranged vertically in the heating chamber, and each of the reaction tubes comprises an at least partial packing of a fluidizable material to form a fluidized bed, each fluidized bed having a L/D-ratio of its length L and its diameter D from 3 to 30,
at least one entry point for gaseous reactants for each reaction tube,
at least one exit point for reaction products for each reaction tube and
at least one heating apparatus for externally heating the reaction tubes,
wherein the apparatus can be divided into segments which can be switched between production mode and regeneration mode independently of one another.

12. The apparatus according to claim 11, wherein the apparatus is of modular construction such that every reaction tube can be individually brought on- and offline for the endothermic reaction.

13. The apparatus according to claim 11, wherein each of the reaction tubes has a diameter of more than 100 mm.

14. The apparatus according to claim 11, wherein at least two of the reaction tubes are connected to one another.

15. A method for carrying out a non-oxidative dehydroaromatization of one or more C₁ to C₄ aliphatics, wherein the method is performed in the apparatus of claim 11.

16. The method according to claim 1, wherein a) comprises introducing between 50 MW and 500 MW of power.

17. The apparatus according to claim 11, wherein each of the reaction tubes has a diameter of from 125 mm to 1500 mm.
